(19) United States
(12) Patent Application Publication
(10) Pub. No.: US 2023/0000968 A1
(43) Pub. Date: Jan. 5, 2023

(54) Recombinant Human Papillomavirus Vaccine Composition and Use thereof

(71) Applicant: IMMUNE-PATH BIOTECHNOLOGY (SUZHOU) CO., LTD., Jiangsu (CN)

(72) Inventors: Li SHI, Shanghai (CN); Zhi ZHANG, Shanghai (CN); Wenli TIAN, Shanghai (CN); Weiliang XUE, Shanghai (CN); Xiaodong SUN, Shanghai (CN); Wei YANG, Shanghai (CN); Wei LI, Shanghai (CN)

(73) Assignee: IMMUNE-PATH BIOTECHNOLOGY (SUZHOU) CO., LTD., Jiangsu (CN)

(21) Appl. No.: 17/623,862

(22) PCT Filed: Jun. 24, 2020
(86) PCT No.: PCT/CN2020/098205

§ 371 Date: Dec. 29, 2021

(30) Foreign Application Priority Data
Sep. 2, 2019 (CN) 201910822785.3

(51) Int. Cl. A61K 39/12; A61K 39/39; A61P 31/20; C12N 7/00

(52) U.S. Cl. CPC A61K 39/12; A61K 39/39; A61P 31/20; C12N 7/00; A61K 2039/55561

(57) ABSTRACT

Disclosed in the present disclosure are a recombinant human papillomavirus vaccine composition and a use thereof. Compared with other combinations of antigens and adjuvants, the new vaccine composition provided in the present invention has a more beneficial immune effect.

### TECHNICAL FIELD

The present disclosure pertains to the field of biomedicine. Specifically, the present disclosure relates to a novel recombinant human papillomavirus vaccine composition and use.

### BACKGROUND ART

Papillomavirus (PV) is a pathogen that can infect vertebrates, and more than 300 species have been found currently. More than 200 species can infect humans and are called Human Papillomavirus (HPV), of which more than 40 species can cause human diseases. The capsid of HPV is composed of a major capsid protein L1 and a minor capsid protein L2. HPV mainly infects skin and mucosal tissues. In most cases, HPV infection causes no obvious clinical symptoms. However, some persistent HPV infections may cause epithelial hyperplasia of the skin and mucosa, and even cancer. Clinically, these symptoms are manifested as different types of epithelial warts, cervical cancer, anal cancer, vaginal cancer and other malignant tumors. According to the relationship with cancer occurrence, these 40 kinds of HPV can be divided into high-risk type and low-risk type. Among them, HPV 16, 18, 31, 33, 35, 39, 45, 51, 52, 56, 58, and 59 are high-risk types, and the others are low-risk types. Among them, the possibility that HPV 6 and HPV 11 infections cause genital warts and recurrent respiratory papillomatosis is more than 90%.

Worldwide, cervical cancer ranks third among malignant tumors having a high incidence rate in women, and second among women aged 15-44. It is estimated that in 2018, there were approximately 569,000 new cases of cervical cancer in women in the world, with an age-adjusted incidence rate of 13.1/100000, and 311,000 women died of cervical cancer. The evaluation report of the International Agency for Research on Cancer shows that the age-adjusted incidence rate of cervical cancer in China is 10.7/100000. It is estimated that there were approximately 106,000 new cases of cervical cancer in China in 2018, accounting for 18.7% of the world, and approximately 48,000 deaths, accounting for 15.3% of the world. Cervical cancer is the sixth most common cancer among Chinese women, and ranks as the third most common cancer among women aged 15-44, next only to breast cancer and thyroid cancer. The situation of cervical cancer prevention and control in China is still severe and facing huge challenges. The economic burden of cervical cancer patients is very serious, especially in rural areas. It is now known that almost all cervical cancers are associated with HPV infection. 70% of cervical cancers are associated with HPV 16 and 18 infections, and about 20% of cervical cancers are associated with HPV 31, 33, 45, 52, and 58 infections.

Administration of preventive HPV vaccine is an effective and feasible approach for preventing cervical cancer, and is a primary prevention measure for cervical cancer. The main measures for secondary prevention of cervical cancer include regular cervical-cancer screening (CCS) for all women of the right age and treating patients with precancerous lesions of cervical cancer as soon as possible. The main measures for tertiary prevention of cervical cancer include appropriate surgery, radiotherapy, chemotherapy and palliative therapy according to the clinical stage. Currently, three preventive HPV vaccines have been marketed globally, namely, bivalent HPV vaccine Cervarix available from GlaxoSmithKline, and 4-valent HPV vaccine Gardasil and 9-valent HPV vaccine Gardasil 9 available from MSD. These three commercially available vaccines are all vaccines based on virus-like particles (VLP) formed by the major capsid protein L1 of HPV as antigens. The L1 protein expressed through genetic recombination can form virus-like particles (VLPs) under particular condition. The adjuvant of Cervarix is aluminum hydroxide adjuvant contains MPL, and the adjuvant of Gardasil and Gardasil 9 is aluminum phosphate adjuvant.

A number of rigorous randomized clinical studies have proved that the immunogenicity after administration of 2 doses of HPV vaccine in women aged 9-14 is not inferior to that in the case of administration of 3 doses of HPV vaccine. In the 2017 WHO position paper, recommended is use of 2-dose immunization procedure (months 0 and 6) in population aged 9-14, which provides a good reference and guiding significance for reducing the number of immunization doses (2 doses) in adult women. In addition, the antibody titer produced by administration of 1 dose of Cervarix can still reach more than 9 times the natural infection level after 7 years. Also in a clinical study in India, it is observed that 870 people aged 10-18 who received 1 dose of Gardasil shows no persistent HPV 16 and 18 infections occurred during an average follow-up of 4.7 years. Although both of these studies are non-rigorously randomized clinical studies with a main purpose of evaluating the protective efficacy of a single-dose HPV vaccine, these findings provide a conceptual basis and a possibility of future realization for a single-dose HPV vaccine. The WHO currently calls for elimination of cervical cancer worldwide. The medical industry believes that an efficient and convenient 9-valent HPV vaccine is needed for achieving this goal. In the three HPV vaccines that are currently commercially available, only Cervarix uses a combined adjuvant (aluminum hydroxide and MPL), and shows high immunogenicity, but it is a bivalent HPV vaccine. Administration procedure of the 9-valent HPV vaccine that is currently commercially available at different age stages is 3 doses for immunization to (months 0, 2 and 6). Therefore, a novel 9-valent HPV vaccine that uses a simplified immunization procedure (1 or 2 doses) is an urgent need, which helps to achieve the elimination of cervical cancer worldwide, and at the same time, greatly alleviates the pressure caused by insufficient production capacity and low vaccination finishing rate, so as to reduce the morbidity and mortality of cervical cancer in the world, especially in developing countries, as soon as possible. Thereby, the plan of elimination of cervical cancer worldwide is promoted, and also good economic and social benefits are produced.

Some studies believe that Cervarix can produce higher antibody titer compared to Gardasil possibly due to the enhanced immune activation by TLR4 agonist MPL in the dual adjuvant of Cervarix, which may also be an important reason why Cervarix can maintain a higher antibody level for a long time. Therefore, it is very important to choose a suitable combined adjuvant to develop the above-mentioned novel 9-valent HPV vaccine that simplifies immunization procedure. The dual adjuvant of Cervarix available from GlaxoSmithKline contains MPL and aluminum hydroxide adjuvant. However, MPL is currently a chemically-treated and attenuated Salmonella lipopolysaccharide, of which the production process is complex and costly, and the production capacity is thus easily limited by the source of MPL. In addition, the conditions for large-scale domestic production of this product are immature and cannot meet the needs of subsequent clinical application. The combined adjuvant system of an aluminum adjuvant and CpG ODN is chosen because, firstly, CpG ODN can be produced with a large scale through chemical synthesis, for which quality control is easy, and secondly, CpG ODN is an to agonist of Toll-like receptor 9 (TLR9), which can stimulate cells expressing TLR9 and activate downstream pathways in innate immune response. CpG ODN, on the one hand, induces the expression of type I interferon and inflammatory factors, and on the other hand, matures plasmacytoid dendritic cells, thereby enhancing humoral and cellular immune responses. An aluminum adjuvant is by far the most widely used human vaccine adjuvant, and a combined adjuvant composed of an aluminum adjuvant and another adjuvant also has a wide range of applications. The CpG ODN adjuvant is a new type of adjuvant approved for use in commercially available vaccines (hepatitis B vaccine product from Dynavax) in addition to aluminum adjuvants. In addition, many vaccines such as hepatitis B and malaria vaccines under development also use the combined adjuvant of an aluminum adjuvant and CpG ODN in clinical trials. It is expected that addition of the CpG ODN adjuvant allows the novel 9-valent HPV vaccine to exhibit better immunogenicity and immune durability than those of the vaccines having a traditional aluminum adjuvant alone, so as to improve the immune prevention effect, for example, to reduce the number of times of immunization, shorten the interval between immunizations, and possibly reduce antigen dosage for immunization.

Although some HPV vaccines have been developed in the prior art worldwide, there are still problems such as low vaccination finishing rate caused by need of relatively more doses of vaccination. Therefore, there is a need to develop improved HPV vaccine products in the art. From a strategic point of view, there is always a need for a new vaccine formulation with improved immunogenicity, for example, improved by attempts and improvements for adjuvant or formulation combinations to increase immune response, reduce number of times of immunization, shorten interval between immunizations, and reduce antigen dosage for immunization.

### SUMMARY

In one aspect of the present disclosure, provided is a recombinant human papillomavirus vaccine, which contains an antigen against human papillomavirus (HPV). More specifically, the aforementioned antigen may be various types of L1 VLP proteins of HPV (HPV 16L1, 18L1, 6L1, 11L1, 31L1, 33L1, 45L1, 52L1, 58L1).

The HPV in the present disclosure is a double-stranded DNA virus. Its genome contains about 8000 base pairs. According to their corresponding expression time, they can be divided into early region (encoding early genes E1, E2, E4, E5, E6, and E7), late region (encoding late genes L1 and L2), and upstream regulatory region (URR, not encoding protein but regulating gene transcription). The capsid of HPV is composed of a major capsid protein L1 and a minor capsid protein L2. Commercially available vaccines are all vaccines based on HPV L1 virus-like particles (VLP) as antigens, in which the L1 protein expressed through genetic recombination can form virus-like particles under particular condition, which has good immunogenicity.

In the NCBI database, many existing sequences of various types of L1 VLP proteins of HPV (HPV 16L1, 18L1, 6L1, 11L1, 31L1, 33L1, 45L1, 52L1, and 58L1) can be selected by those skilled in the art. These sequences can be used as an ideal selection basis for antigen protein. For the purpose of developing the present disclosure, in the examples of the present disclosure, the inventors mostly use highly conserved sequences in the prior art, specifically including: the amino acid sequence of HPV 6L1 recorded at the NCBI database in 1995 with accession number AAA74218, the amino acid sequence of HPV 11L1 recorded at the NCBI database in 1994 with accession number AAA46935; the amino acid sequence of HPV 16L1 recorded at the NCBI database in 1998 with accession number AAC09292.1; the amino acid sequence of HPV 18L1 protein recorded at the NCBI database in 2003 with accession number AAQ92369.1, the amino acid sequence of HPV 31L1 protein recorded at the NCBI database in 1994 with accession number AAA46956; the amino acid sequence of HPV 33L1 protein recorded at the NCBI database in 2009 with accession number ACL12333.1, the amino acid sequence of HPV 45L1 protein recorded at the NCBI database in 2009 with accession number ABP99831.1 (in which the N-terminal 26 amino acids are truncated because the above 26 amino acids constitute a hydrophobic region, which may affect the formation of VLPs from L1 protein); the amino acid sequence of HPV 52L1 protein recorded at the NCBI database in 2005 with accession number CAA52590.1 (in which the N-terminal 27 amino acids are truncated because the above 27 amino acids constitute a hydrophobic region, which may affect the formation of VLPs from L1 protein); and the amino acid sequence of HPV 58L1 protein recorded at the NCBI database in 2009 with accession number CAX48979.1.

The above-mentioned antigens can be easily obtained by conventional technical means of modern molecular biology. Typical methods include: expressing the above various types of L1 VLP proteins of HPV in Pichia pastoris, which includes the steps of:

(1) cloning genes (that are codon optimized) of various types of L1 protein of HPV of the present disclosure into the expression vector;

(2) transforming the expression vector obtained in step (1) into Pichia pastoris host strains;

(3) obtaining strains stably expressing various types of L1 proteins of HPV through strain screening; and

(4) using the strains obtained in step (3) for expression to obtain various types of L1 proteins of HPV.

The protein obtained above is subjected to a conventional processing method, such as hydrophobic chromatography, anion exchange chromatography, or hydroxyapatite chromatography, to obtain an antigen protein with a relatively high purity.

It should be noted that the method of stably expressing various types of L1 proteins of HPV using Pichia pastoris expression system is a well-known method in the art, specifically, referring to “Molecular Cloning: A Laboratory Manual” and other literatures. Those skilled in the art may also choose other expression methods such as Escherichia coli, Saccharomyces cerevisiae, Hansenula polymorpha, CHO cells, and insect cells to obtain various types of L1 proteins of HPV.

In a preferred embodiment, the vaccine composition further includes a pharmaceutically acceptable system (adjuvant). In a preferred embodiment, the pharmaceutically acceptable system is a combined adjuvant of an aluminum adjuvant and CpG ODN. The inventors of the present disclosure found that the combination of the above two adjuvants and various types of L1 proteins of HPV shows significantly improved immunological activity. Therefore, the vaccine formulation composition of the present disclosure becomes a new generation of recombinant human papillomavirus vaccine.

The aluminum adjuvant of the present disclosure is currently the most widely used type of adjuvant in vaccines. It has a history of more than 80 years of application and has been used by tens of billions of people. It was once the unique adjuvant approved by the U.S. Food and Drug Administration (FDA) for use in human vaccines. At present, there have been many studies on the use of aluminum adjuvants in vaccines. More systematic studies can be found in, for example, Chapter 8 “Adjuvant properties of aluminum and calcium compounds” and Chapter 9 “Structure and properties of aluminum adjuvants” in the book “Vaccine Design: The Subunit and Adjuvant Approach” (ISBN: 0-306-44867-X), or for example Chapter 4 “Use of aluminum compounds as vaccine adjuvants” in the book “Vaccine Adjuvants: Preparation Methods and Research Protocols” (ISBN: 1-59259-083-7). There have been some commercial aluminum adjuvants available for use in vaccines, including but not limited to Alhydrogel® (aluminum hydroxide) and Adju-Phos® (aluminum phosphate). The foregoing studies have also described preparation methods of preparing aluminum adjuvants, and those skilled in the art can thus prepare a desired aluminum adjuvant and apply it in vaccine formulations as needed.

The CpG ODN adjuvant is a class of immunostimulatory oligonucleotides known to have adjuvant properties, and can activate B cells, NK, dendritic cells (DC), etc. and induce the release of IL-12 and IFN-γ, thereby inducing a strong Th1-type response and cellular immunity. This type of adjuvant is also well-known to those skilled in the art, and has been described in the prior art, for example, in international applications such as WO 96/02555 and WO 99/33488. There are currently some commercialized CpG ODN adjuvants, such as CpG 7909 (Coley) or CpG 1018 (Dynavax).

The specific preparation method of the vaccine formulation provided by the present disclosure can refer to, for example, the book “Vaccine Design: The Subunit and Adjuvant Approach” (ISBN: 0-306-44867-X). As one of the most conventional methods, the preparation method provided by the present disclosure involves mixing the above antigen protein of the present disclosure with the above pharmaceutically acceptable system (carrier) adjuvant. As a more specific example, in a preferred embodiment, VLPs of various types of L1 proteins of HPV are adsorbed onto an aluminum adjuvant to maintain the stability of the VLP and enhance its immunogenicity.

Regarding the principle of selecting antigen dosage of vaccine formulations, the dosage of each vaccination should be able to cause an immune protective response in the vaccinated person without significant toxic side effects. Generally, the dosages of different antigen proteins are slightly different, and the optimal dosage of a particular vaccine can be determined by observing the antibody titer and other reactions of the subject. In the vaccine formulation provided by the present disclosure, the content of the antigen, various types of L1 VLP proteins of HPV, is between 2-60 μg, the contents of the specific subtypes HPV 16L1, 18L1, 6L1, 11L1, 31L1, 33L1, 45L1, 52L1, and 58L1 protein are respectively 6-60 μg, 4-40 μg, 3-30 μg, 4-40 μg, 2-20 μg, 2-20 μg, 2-20 μg, 2-20 μg, and 2-20 μg, the content of the aluminum adjuvant is about 225-1000 μg and more preferably 500 μg, and the content of the CpG ODN adjuvant is approximately 250-1000 μg and more preferably 500-1000 μg.

In a second aspect of the present disclosure, use of the above vaccine composition is provided for preventing or treating diseases or disorders associated with human papillomavirus infection.

In a third aspect of the present disclosure, provided is a test vaccine kit including a vaccine administration device, including but not limited to a syringe device, a liquid ejection device, a powder device, and a nebulizer device. The selection of the devices mainly depends on modes of administration. Common modes of administration include intramuscular injection, intraperitoneal injection, intradermal injection or subcutaneous injection, or oral/digestive tract, respiratory tract and genitourinary tract mucosal administrations. The vaccine of the present disclosure may usually be injected intramuscularly, and the common administration device is a syringe device. Commonly, the vaccine can be administered as a single dose, or its components can also be administered in combination at the same time or at different times.

Specifically, the present disclosure provides the following specific embodiments.

1. A recombinant human papillomavirus (HPV) vaccine formulation composition, comprising at least VLPs formed by assembly of major capsid to proteins L1 of the following nine types of HPV: HPV 16L1, 18L1, 6L1, 11L1, 31L1, 33L1, 45L1, 52L1, and 58L1 and a pharmaceutically acceptable combined adjuvant system.

2. The vaccine formulation composition according to embodiment 1, wherein the pharmaceutically acceptable combined adjuvant system is a combined adjuvant of an aluminum salt adjuvant and a CpG ODN.

3. The vaccine formulation composition according to embodiment 1, wherein a content of VLPs formed by assembly of L1 of each HPV type is between 2-60 μg.

4. The vaccine formulation composition according to embodiment 3, wherein contents of VLPs formed by assembly of HPV 16L1, 18L1, 6L1, 11L1, 31L1, 33L1, 45L1, 52L1, and 58L1 are respectively 6-60 μg, 4-40 μg, 3-30 μg, 4-40 μg, 2-20 μg, 2-20 μg, 2-20 μg, 2-20 μg, and 2-20 μg.

5. The vaccine formulation composition according to embodiment 2, wherein the aluminum adjuvant is selected from aluminum hydroxide and aluminum phosphate.

6. The vaccine formulation composition according to embodiment 5, wherein the aluminum adjuvant is aluminum phosphate.

7. The vaccine formulation composition according to embodiment 5, wherein the aluminum phosphate has a PI value of 5 to 9.5.

8. The vaccine formulation composition according to embodiment 2, wherein a content of the aluminum adjuvant is approximately 225-1000 μg.

9. The vaccine formulation composition according to embodiment 8, wherein the content of the aluminum adjuvant is approximately 500 μg.

10. The vaccine formulation composition according to embodiment 2, wherein a content of the CpG ODN is approximately 200-1500 μg.

11. The vaccine formulation composition according to embodiment 10, wherein the content of the CpG ODN is approximately 250-1000 μg.

12. The vaccine formulation composition according to embodiment 2, wherein contents of VLPs formed by assembly of HPV 16L1, 18L1, 6L1, 11L1, 31 L1, 33L1, 45L1, 52L1, and 58L1 are respectively 6-60 μg, 4-40 μg, 3-30 μg, 4-40 μg, 2-20 μg, 2-20 μg, 2-20 μg, 2-20 μg, and 2-20 μg, a content of the aluminum adjuvant is approximately 225-1000 μg, and a content of the CpG ODN is approximately 250-1000 μg.

13. The vaccine formulation composition according to embodiment 12, wherein the contents of VLPs formed by assembly of HPV 16L1, 18L1, 6L1, 11 L1, 31L1, 33L1, 45L1, 52L1, and 58L1 are respectively 6-60 μg, 4-40 μg, 3-30 μg, 4-40 μg, 2-20 μg, 2-20 μg, 2-20 μg, 2-20 μg, and 2-20 μg, the content of the aluminum adjuvant is approximately 500 μg, and the content of the CpG ODN is approximately 500-1000 μg.

14. Use of the vaccine formulation composition according to any one of embodiments 1-13 in preparation of a medicament for preventing or treating human papillomavirus infection-associated diseases, wherein the medicament is administered through 1- or 2-dose vaccination for immunization.

15. A test vaccine kit, comprising the vaccine formulation composition according to embodiments 1, wherein the kit comprises a vaccine administration device selected from a syringe device, a liquid ejection device, a powder device, and a nebulizer device.

The inventors of the present disclosure found that the combination of the above two adjuvants and various types of L1 VLP proteins of HPV shows surprisingly high immunological activity, and can even reduce number of times of immunization, shorten interval between immunizations, and reduce antigen dosage for immunization. Thus, the vaccine composition of the present disclosure becomes a new generation of human papillomavirus vaccine.

Other aspects of the present disclosure are obvious to those skilled in the art due to the disclosure herein.

### DETAILED DESCRIPTION OF THE EMBODIMENTS

### Example 1: Preparation of Vaccine Composition Containing Various Types of L1 VLP Antigen Proteins of HPV

In order to investigate the technical effect of the vaccine formulation composition provided by the present disclosure, the inventors of the present disclosure prepared the following various vaccine formulation compositions (0.5 ml/dose), each of formulation compositions contains various types of L1 VLP proteins of HPV (HPV 16L1, 18L1, 6L1, 11L1, 31L1, 33L1, 45L1, 52L1, and 58L1), an aluminum adjuvant, and a CpG ODN adjuvant. The specific preparation method was as follows: firstly, a stock solution of various types of to L1 VLP antigens of HPV was adsorbed on the aluminum adjuvant (aluminum phosphate adjuvant AP, purchased from Shanghai Zerun Biotechnology Co., Ltd.) to prepare adsorption samples having different ratios of various types of L1 VLP antigens of HPV/aluminum adjuvant (w/w) (here, the aluminum adjuvant content is substantially the content of aluminum element); and then different concentrations of CpG ODN samples (CpG 7909 or CpG 1018, both purchased from Guangzhou RiboBio Co., Ltd.) were added into the antigen/aluminum adsorption samples. After the above formulations were thoroughly mixed, if not administrated immediately, they were stored at 4° C. The specific ratios were as follows.

TABLE 1

| | | | | | | | | | | | aluminum | | |
|---|---|---|---|---|---|---|---|---|---|---|---|---|---|
| | | | | | | | | | | HPV | phosphate | CpG ODN | |
| | various types of L1 VLP of HPV (μg) | | | | | | | | | antigen | adjuvant | adjuvant | volume |
| No. | 16 | 18 | 6 | 11 | 31 | 33 | 45 | 52 | 58 | dosage | (AP)(μg) | (μg) | (μl) |
| 1 | 60 | 40 | 30 | 40 | 20 | 20 | 20 | 20 | 20 | 1X | 500 | 500 | 500 |
| 2 | 60 | 40 | 30 | 40 | 20 | 20 | 20 | 20 | 20 | 1X | 500 | 1000 | 500 |
| 3 | 60 | 40 | 30 | 40 | 20 | 20 | 20 | 20 | 20 | 1X | 500 | 0 | 500 |
| 4 | 6 | 4 | 3 | 4 | 2 | 2 | 2 | 2 | 2 | 0.1X | 500 | 500 | 500 |
| 5 | 6 | 4 | 3 | 4 | 2 | 2 | 2 | 2 | 2 | 0.1X | 500 | 1000 | 500 |
| 6 | 60 | 40 | 30 | 40 | 20 | 20 | 20 | 20 | 20 | 1X | 500 | 1000 | 500 |
| 7 | 6 | 4 | 3 | 4 | 2 | 2 | 2 | 2 | 2 | 0.1X | 500 | 1000 | 500 |

Note:
The CpG ODN contained in vaccines Nos. 1, 2, 4, and 5 are CpG 7909, and the CpG ODN contained in vaccines Nos. 6 and 7 are CpG 1018.

### Example 2

For the obtained recombinant human papillomavirus vaccine formulation compositions of Example 1 to be evaluated, the inventors carried out immunogenicity studies using BALB/c mice as an animal model. The immunogenicity of the vaccine formulation compositions provided by the present disclosure was investigated by using various types of L1 VLP proteins of HPV as antigens and using an aluminum adjuvant and CpG ODN as to adjuvants. 6-8-week-old BALB/c female mice were randomly divided into groups, 10 mice in each group. The vaccines prepared using various types of L1 VLP proteins of HPV in combination with adjuvants (Table 1) were administrated into mice through intramuscular injection, with an injection volume of 0.05 ml (i.e. 1/10 dose). A self-prepared vaccine group, a marketed vaccine Gardasil 9 group, and an adjuvant control group were set. Among them, 1-dose group was immunized on day 0, blood samples were collected on day 35; 2-dose group was immunized on days 0 and 21, and blood samples were collected on days 35 and 56; and 3-dose group was immunized on days 0, 21, and 42, and blood samples were collected on day 56. Pseudovirion-Based Neutralization Assay (PBNA) was used to detect the titers of neutralizing antibodies against various types of L1 VLP proteins of HPV in the serum, and the geometric mean titers (GMT) of the antibodies were calculated.

The immunogenicity evaluation method is a conventional technical means in to the art. As an example, the more specific operation method of the Pseudovirion-Based Neutralization Assay is as follows.

(1) HEK 293FT cells in a good proliferation condition were digested into individual cells with 0.25% Trypsin-EDTA, the cells were diluted to a suitable concentration with a DMEM complete medium, and the cells were counted by a cell counter.

(2) According to the cell count results, HEK 293FT cells were diluted to a density of 1.5×10⁵/ml with the DMEM complete medium and pre-plated in a 96-well cell culture plate, with each well added 100 μl of cell dilution, and incubated in an incubator at 37° C. and 5% CO₂ until cell adhesion.

(3) An inactivated serum sample was diluted with the DMEM complete medium to an appropriate initial dilution, and then several gradients of consecutive two-fold dilutions were performed, with double wells for each dilution.

(4) The pseudovirus solution was diluted to log(TCID₅₀/0.1 ml)=3.2±0.5 with the DMEM complete medium and mixed thoroughly.

(5) Into the serum dilution, equal volume of pseudovirus dilution was added, mixed thoroughly, and incubated at room temperature for 60 minutes. Positive control wells were also set, each well containing the DMEM complete medium and equal volume of pseudovirus dilution; and blank control wells were set, each well containing the DMEM complete medium.

(6) After the incubation, 100 μl of the serum-pseudovirus mixed liquid was accurately taken and added slowly and carefully to the pre-plated 96-well cell to plate.

(7) The resultant was incubated in an incubator at 37° C. and 5%002 for 72 hours, the result was observed using a fluorescence microscope, or a spot analyzer was used to read, analyze, and process the results.

The studying results are shown in Tables 2 to 5 below.

As shown in Table 2, addition of a CpG ODN adjuvant to a traditional 9-valent HPV vaccine containing only an aluminum adjuvant can significantly increase the neutralizing antibody titer in the serum of immunized mice, and GMT was increased by 2.64 to 13.93 times.

TABLE 2
Comparison of neutralizing antibody titers (GMT) in mouse serum on
day 35 after immunization by one dose with each vaccine

| | Group | | |
|---|---|---|---|
| | | Group of Vaccine | |
| HPV | Group of vaccine No. 1 | No. 3 | No. 1/No. 3 |
| Type | HPV1X+AP+CpG | HPV1X+AP | (fold) |
| HPV 16 | 3719 | 4222 | 3.25 |
| HPV 18 | 13719 | 3676 | 3.73 |
| HPV 6 | 2599 | 746.4 | 3.48 |
| HPV 11 | 2425 | 746.4 | 3.25 |
| HPV 31 | 4222 | 649.8 | 6.50 |
| HPV 33 | 4222 | 984.9 | 4.29 |
| HPV 45 | 6859 | 857.4 | 8.00 |
| HPV 52 | 9051 | 3430 | 2.64 |
| HPV 58 | 19401 | 1393 | 13.93 |

Note:
The formulas of vaccines Nos. 1 and 3 are shown in Table 1. Vaccine No. 3 does not contain CpG ODN, and the contents of the other components are the same as those of vaccine No

As shown in Table 3, the level of neutralizing antibodies on day 35 after immunization by one dose of vaccine No. 1 containing the combined adjuvant was compared with that on day 14 after immunization by two doses of the marketed vaccine Gardasil 9 (Merck). GMTs of HPV 16, 6, 11, and 52 were substantially equivalent to that for Gardasil 9, and GMTs of the other five types were better than that for Gardasil 9.

TABLE 3
Comparison of neutralizing antibody titers GMT in mouse serum on
day 35 after immunization by one dose of the novel 9-valent HPV
vaccine and day 14 after immunization by two doses of Gardasil 9

| | Group | | |
|---|---|---|---|
| | Vaccine No. 1 | Gardasil 9 | |
| | 1 × HPV + AP + CpG | 1 × HPV + AP | No. 1/ |
| HPV | on day 35 after one | on day 14 after | Gardasil 9 |
| Type | dose | two doses | (fold) |
| HPV 16 | 13719 | 20794 | 0.66 |
| HPV 18 | 13719 | 7611 | 1.80 |
| HPV 6 | 2599 | 3430 | 0.76 |
| HPV 11 | 2425 | 1656 | 1.46 |
| HPV 31 | 4222 | 2425 | 1.74 |
| HPV 33 | 4222 | 828.2 | 5.10 |
| HPV 45 | 6859 | 509.8 | 13.45 |
| HPV 52 | 9051 | 11943 | 0.76 |
| HPV 58 | 19401 | 11143 | 1.74 |

Note:
The formula of vaccine No. 1 is shown in Table 1; the marketed vaccine Gardasil 9 does not contain CpG ODN, and the antigen dosage is the same as that of vaccine No. 1, that is, the antigen dosage of Gardasil 9 is the same as the 1 × antigen dosage of the present disclosure; and each group has 10 mice.

As shown in Table 4, the neutralizing antibody level on day 35 after immunization by two doses of vaccine No. 1 containing the combined adjuvant was compared with that on day 14 after immunization by three doses of the marketed vaccine Gardasil 9. The GMTs of HPV 6, 11, 31, and 52 were substantially equivalent to that for Gardasil 9, and the GMTs of the other five to types were better than that for Gardasil 9. In accordance with the comparison data of one dose to two doses in Table 3, the above results show that the novel 9-valent HPV vaccine containing the combined adjuvant provides better immunogenicity even if the number of doses for immunization is reduced.

TABLE 4
Comparison of neutralizing antibody titers GMT in mouse serum after
immunizatior by two doses of the novel 9-valent HPV vaccine and after
immunization by three doses of Gardasil 9

| | Group | | |
|---|---|---|---|
| | Vaccine No. 1 | Gardasil 9 | |
| | 1 × HPV + AP + CpG | 1 × HPV + AP | |
| HPV | on day 35 after two | on day 14 after | No. 1/Gardasil 9 |
| Type | doses | three doses | (fold) |
| HPV 16 | 81275 | 38802 | 2.09 |
| HPV 18 | 32254 | 11143 | 2.89 |
| HPV 6 | 14368 | 11143 | 1.29 |
| HPV 11 | 5080 | 2986 | 1.70 |
| HPV 31 | 6400 | 5572 | 1.15 |
| HPV 33 | 18102 | 1715 | 10.56 |
| HPV 45 | 32254 | 606.3 | 53.20 |
| HPV 52 | 18102 | 14703 | 1.23 |
| HPV 58 | 36204 | 16890 | 2.14 |

Note:
The formula of vaccine No. 1 is shown in Table 1; the marketed vaccine Gardasil 9 does not contain CpG ODN, and the antigen dosage is the same as that of vaccine No. 1, that is, the antigen dosage of Gardasil 9 is the same as the 1 × antigen dosage of the present disclosure; and each group has 10 mice.

As shown in Table 5, the neutralizing antibody level on day 35 after immunization by one dose of vaccine No. 1 containing the combined adjuvant was compared with that on day 14 after immunization by two doses of the marketed vaccine Gardasil 9. The GMTs of HPV 16, 6, and 52 were substantially the same as those of Gardasil 9, and the GMTs of the other six types were better than those of Gardasil 9.

As shown in Table 5, in a case where the CpG ODN adjuvant was added to the traditional 9-valent HPV vaccine containing only an aluminum adjuvant, even if the HPV antigen content was reduced to 1/10 dosage of Gardasil 9, the to neutralizing antibody levels of the nine HPV types was still comparative to those for Gardasil 9.

TABLE 5
Comparison of neutralizing antibody titers GMT in mouse serum on
day 35 after immunization by one dose of each vaccine

| | Group | | |
|---|---|---|---|
| HPV | Vaccine No. 5 | Group Gardasil 9 | No. 5/Gardasil 9 |
| type | 0.1 × HPV + AP + CpG | 1 × HPV + AP | (fold) |
| HPV 16 | 22286 | 19401 | 1.15 |
| HPV 18 | 6400 | 5572 | 1.15 |
| HPV 6 | 2111 | 800 | 2.64 |
| HPV 11 | 1393 | 800 | 1.74 |
| HPV 31 | 2786 | 1600 | 1.74 |
| HPV 33 | 2786 | 1393 | 2.00 |
| HPV 45 | 3676 | 800 | 4.60 |
| HPV 52 | 1393 | 2111 | 0.66 |
| HPV 58 | 6400 | 8445 | 0.76 |

Note:
The formula of vaccine No. 5 is shown in Table 1. The marketed vaccine Gardasil 9 does not contain CpG ODN, and the aluminum adjuvant content is the same as that of vaccine No. 5. The dosage of 9 HPV types is 10 times that of vaccine No. 5, that is, the antigen dosage of Gardasil 9 is the same as the 1 × antigen dosage in the present disclosure.

### Example 3

The inventors also investigated the immunogenicity of the novel 9-valent HPV vaccines with different CpG ODN contents (such as 500-1000 μg) and the immunogenicity of the novel 9-valent HPV vaccines containing different CpG to ODN types (such as CpG7909 and CpG1018). The results show that the novel 9-valent HPV vaccines of various formulations all have good immunogenicity. The experimental procedure in mice and neutralizing antibody detection method are the same as those in Example 2. The studying results are shown in Table 6 below.

TABLE 6
Neutralizing antibody titers GMT in mouse serum on day 14 after
immunization by two doses of each vaccine

| | Group | | | |
|---|---|---|---|---|
| | Vaccine No. 2 | Vaccine No. 4 | Vaccine No. 6 | Vaccine No. 7 |
| HPV | 1 × HPV + AP + | 0.1 × HPV + AP + | 1 × HPV + AP + | 0.1 × HPV + AP + |
| type | 1000CpG7909 | 500CpG7909 | 1000CpG1018 | 1000CpG1018 |
| HPV 16 | 38802 | 44572 | 16890 | 11143 |
| HPV 18 | 22286 | 11143 | 8445 | 9701 |
| HPV 6 | 4850 | 3676 | 1600 | 1056 |
| HPV 11 | 1838 | 1838 | 606.3 | 919 |
| HPV 31 | 4850 | 2111 | 527.8 | 527.8 |
| HPV 33 | 29407 | 11143 | 7352 | 2786 |
| HPV 45 | 4850 | 6400 | 9701 | 4850 |
| HPV 52 | 1393 | 1838 | 919 | 696.4 |
| HPV 58 | 14703 | 11143 | 6400 | 8445 |

Note:
the formulas of vaccines Nos. 2, 4, 6, and 7 vaccine are shown in Table 1. The mouse muscle immunization dosage is 1/10 dosage, and the immunization volume is 0.05 ml.

It can be seen from Tables 2 to 6 that the vaccine compositions of the present disclosure prepared according to the specific component and ratio have good immunological activity, especially when the contents of the various types of L1 VLP proteins of HPV (HPV 16L1, 18L1, 6L1, 11L1, 31L1, 33L1, 45L1, 52L1, and 58L1) are respectively 6-60 μg, 4-40 μg, 3-30 μg, 4-40 μg, 2-20 μg, 2-20 μg, 2-20 μg, 2-20 μg, and 2-20 μg, the content of the aluminum adjuvant is 500 to μg, and the content of the CpG ODN adjuvant is 500-1000 μg, the compositions have excellent immune effects, and can be used as a new generation of vaccine formulation composition and as a guide for dosage selection ranges of the antigen and combined adjuvant in the formulation.

All documents mentioned in the present disclosure are cited as references in this application, as if each document was individually cited as a reference. In addition, it should be understood that after reading the above teaching content of the present disclosure, those skilled in the art can make various changes or modifications to the present disclosure, and these equivalent forms also fall within the scope defined by the appended claims of the present application.

1. A recombinant human papillomavirus (HPV) vaccine formulation composition, comprising 9 types of VLPs, each formed by the assembly of one of the 9 types of major capsid proteins: HPV 16L1, 18L1, 6L1, 11L1, 31L1, 33L1, 45L1, 52L1, and 58L1, and a pharmaceutically acceptable combined adjuvant system.

2. The vaccine formulation composition according to claim 1, wherein the pharmaceutically acceptable combined adjuvant system is a combination of aluminum salt adjuvant and a CpG ODN adjuvant.

3. The vaccine formulation composition according to claim 1, wherein a content of L1 protein VLPs of each HPV type is between 2-60 μg.

4. The vaccine formulation composition according to claim 3, wherein contents of VLPs for HPV 16L1, 18L1, 6L1, 11L1, 31L1, 33L1, 45L1, 52L1, and 58L1 types are respectively 6-60 μg, 4-40 μg, 3-30 μg, 4-40 μg, 2-20 μg, 2-20 μg, 2-20 μg, 2-20 μg, and 2-20 μg.

5. The vaccine formulation composition according to claim 2, wherein the aluminum adjuvant is selected from aluminum hydroxide and aluminum phosphate.

6. The vaccine formulation composition according to claim 5, wherein the aluminum adjuvant is aluminum phosphate.

7. The vaccine formulation composition according to claim 5, wherein the aluminum phosphate has a PI value of 5 to 9.5.

8. The vaccine formulation composition according to claim 2, wherein a content of the aluminum adjuvant is approximately 225-1000 μg.

9. The vaccine formulation composition according to claim 8, wherein the content of the aluminum adjuvant is approximately 500 μg.

10. The vaccine formulation composition according to claim 2, wherein a content of the CpG ODN is approximately 200-1500 μg.

11. The vaccine formulation composition according to claim 10, wherein the content of the CpG ODN is approximately 250-1000 μg.

12. The vaccine formulation composition according to claim 2, wherein contents of VLPs for HPV 16L1, 18L1, 6L1, 11L1, 31L1, 33L1, 45L1, 52L1, and 58L1 types are respectively 6-60 μg, 4-40 μg, 3-30 μg, 4-40 μg, 2-20 μg, 2-20 μg, 2-20 μg, 2-20 μg, and 2-20 μg, a content of the aluminum adjuvant is approximately 225-1000 μg, and a content of the CpG ODN is approximately 250-1000 μg.

13. The vaccine formulation composition according to claim 12, wherein the contents of VLPs for HPV 16L1, 18L1, 6L1, 11L1, 31L1, 33L1, 45L1, 52L1, and 58L1 types are respectively 6-60 μg, 4-40 μg, 3-30 μg, 4-40 μg, 2-20 μg, 2-20 μg, 2-20 μg, 2-20 μg, and 2-20 μg, the content of the aluminum adjuvant is approximately 500 μg, and the content of the CpG ODN is approximately 500-1000 μg.

14. (canceled)

15. A test vaccine kit, comprising the vaccine formulation composition according to claim 1, wherein the kit comprises a vaccine administration device selected from a syringe device, a liquid ejection device, a powder device, and a nebulizer device.

16. The vaccine formulation composition according to claim 2, wherein the CpG ODN adjuvant is CpG7909.
